SEQUENCE LISTING (20 sequences)

SEQ ID NO: 1 - DNA, 2321 bp, Rattus norvegicus
tgatcacgaaggtcagtttggaaaattaaaattagaatatgtatggacggatggtgacagggagggagtgtaatatttattaacttgttgacgtaactacccttccttccggttcatctccggtcacgatttcaagggtaacttagttatgtgagtttgacttaaattacgaatagactcgtgggtatagtggaggattgtgttacggataattcttcagataaacggggaggaccatacacctcgttctcttcccttgaaatgtcttacgaagacctaataatattccgagtagaagtaccacgggaaaggagattcttggtctcaatcctctcacctccatctcaatatcttgttaaatgaccttttttttaccctaccaatgaagattgtatcgtcttcggttcattcctcctctgttctctatgtctctctttcaagatcgttctgagaccacgatacaaatgccgaaaatcagtagcatttgtagttgacgtcctgggagatgaatccctacgaagtgtttgaccatcagattcttttccttcttctctctccctcccctttttactctctactctcctctcccttttacctgttgtccttcttaccttgaactgttttgtaaatcatttccacaccgactgtgtgccagcgtcagtatttctcgttagctcgagacgcgattcggtgagatgtgtaacttcgtgttagccacgttaactcaatcaagacggtctcgacctacctcgacctgtggaaagagacgagaaagtggggtagacgacctagacggttctcgacgggactctagttctgtggtcgtcgtggttactctttcacttaatctattaaatatgtcgacactgaagtcggattagacgattctttgaaggacccccggcactattttacgtgtaagactaaggtatttggtctggactcggtactctacaaggtaaaaattatcctggagtaccttcgactagggacctcgattgagatccatggtcccgaaatatagtagttactgttgattgcgcttttgggagtcattccagagggttgactgtccctttcaccaaggataaagaattacttcgaggtacaggtggtcaatcaagttacgtgtaagacttcttcatttaagtaagtctcatgaggaggtcaacaaagttacccgataagggtattttcaatagttaatatggtacctcaactatgtgaacctgtgggtctaaatcgaccctttacgacaccactagaacagtatatcttatactcaaagtaaaatttgtttacaaatgtgtttagttacgaagagtgtttagtcgttgtctcagggtttctgacacaccgaaccggtgccagagtatcggataactactatcgtcccttctttttagatgaatactaggggcctcaaagaacgacagacaagtgtgtaggaccaataaagggttaaaataaaggaagacaactttaccggtcttaacatagacgatgtatgatattaatcattcctggatatatgcacgaccttcaatgtctttcattgtcgtacgtagagtctggttatcaaacggagggtacagaaagaaacataagattacagtgtggggaagagtcactttaatctaaattttgtaaagctttctactttttctttattatctgggaaacgatagtcgtagtcactgcaacccataagagtaaaacggaagacacagagtaattgtatcacgtgtaatgtaaaattaactgtaaattttagaggggtgtttctctcatgtttaagtaaggtatgtttcatctactcacccaataactatacacacgtgacccactcgctaaacaaacggatctacttaccaggtagtcaataagtccttgagtcctaccggtttatcgtgtagttccctgttctgggagtttactgaggagatccgagtaaactgcagttttaagtgaagtctctcgacttcatcaaaaagtgcggactgactctaacttcccacataaaggtttgatgggaaaggtaagggtttgtctttccgtgttctcatttatttttcaaaggcccggagactccagagtggggccatagtcgcactcctcgtctactcgtctcacccgctcactttagtaaacttgttttggggtaataaatgtctactctttaaatataacagtcgcattatagacgttccgatttgtcggacctctgatatattttcgtgtcgtgggaccacgtcttcccgtctcgaacccgcgtcttgtgtgagtttcaagggtttcctcgaggtggacagatgttcgaa

SEQ ID NO: 2 - DNA, 1828 bp, Homo sapiens
gtcgacaggctgcatcagaagaggccatcaagcaggtctgttccaagggcctttgcgtcaggtgggctcaggattccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaactgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagccctgctgtgccgtctgtgtgtcttgggggccctgggccaagccccacttcccggcactgttgtgagcccctcccagctctctccacgctctctgggtgcccacaggtgccaacgccggccaggcccagcatgcagtggctctccccaaagcggccatgcctgtcggctgcctgctgcccccaccctgtggctcagggtccagtatgggagctgcgggggtctctgaggggccaggggtggtggggccactgagaagtgacttcttgttcagtagctctggactcttggagtccccagagaccttgttcaggaaagggaatgagaacattccagcaattttccccccacctagccctcccaggttctatttttagagttatttctgatggagtccctgtggatcc

SEQ ID NO: 3 - DNA, 1196 bp, Artificial
  Neomycin resistance from pCDNA3
cagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgta

SEQ ID NO: 4 - DNA, 26 bp, Rattus norvegicus
atgagaaagcttgtagacaggtggag

SEQ ID NO: 5 - DNA, 26 bp, Rattus norvegicus
acaacactagtgcttccagtcaaacc

SEQ ID NO: 6 - DNA, 26 bp, Homo sapiens
aagttgtcgacaggctgcatcagaag

SEQ ID NO: 7 - DNA, 25 bp, Homo sapiens
ataggatccacagggactccatcag

SEQ ID NO: 8 - DNA, 25 bp, Artificial
  primer used for sequencing of GLP-1/Ins/pbud plasmid
gacgtcaaaattcacttcagagagc

SEQ ID NO: 9 - DNA, 22 bp, Artificial
  primer used for sequencing of GLP-1/Ins/pbud plasmid
gctaaatctgggtgtccaagtg

SEQ ID NO: 10 - DNA, 22 bp, Artificial
  primer used for sequencing of GLP-1/Ins/pbud plasmid
aagctccatgtccaccagttag

SEQ ID NO: 11 - DNA, 20 bp, Artificial
  primer used for sequencing of GLP-1/Ins/pbud plasmid
ctcacggcagctccatagtc

SEQ ID NO: 12 - DNA, 21 bp, Artificial
  primer used for sequencing of GLP-1/Ins/pbud plasmid
tgttccacaatgccacgcttc

SEQ ID NO: 13 - DNA, 23 bp, Artificial
  forward primer to amplify neomycin resistant gene
gaattccagaagtagtgaggagg

SEQ ID NO: 14 - DNA, 25 bp, Artificial
  reverse primer to amplify neomycin resistant gene
tctagatacattgatgagtttggac

SEQ ID NO: 15 - DNA, 25 bp, Rattus norvegicus
gaattcgagctgagaggaggtgtag

SEQ ID NO: 16 - DNA, 25 bp, Rattus norvegicus
ctcgagatacctgcctaccactgtc

SEQ ID NO: 17 - DNA, 21 bp, Artificial
  primer for beta-actin RT-PCR
gtgtgatggtgggaatgggtc

SEQ ID NO: 18 - DNA, 20 bp, Artificial
  primer for beta-actin RT-PCR
aggaagaggatgcggcagtg

SEQ ID NO: 19 - DNA, 21 bp, Artificial
  primer for beta-actin RT-PCR
ggcacattcaccagcgactac

SEQ ID NO: 20 - DNA, 20 bp, Artificial
  primer for beta-actin RT-PCR
caatggcgacttcttctggg

(19) United States
(12) Patent Application Publication
(10) Pub. No.: US 2014/0112895 A1
(43) Pub. Date: Apr. 24, 2014

(54) GLP-1 PROMOTER MEDIATED INSULIN EXPRESSION FOR THE TREATMENT OF DIABETES

(71) Applicants: Zalinah Haji Ahmad, Serdang (MY); Abdul Rahman Omar, Serdang (MY)

(72) Inventors: Zalinah Haji Ahmad, Serdang (MY); Abdul Rahman Omar, Serdang (MY)

(73) Assignee: UNIVERSITI PUTRA MALAYSIA, Selamgor (MY)

(21) Appl. No.: 13/825,085

(22) PCT Filed: Oct. 5, 2011
(86) PCT No.: PCT/MY2011/000215

§ 371 Date: Aug. 8, 2013

(30) Foreign Application Priority Data
Oct. 8, 2010 (MY) PI 2010700074

(51) Int. Cl. C12N 15/85

(52) U.S. Cl. CPC C12N 15/85
USPC 424/93.21; 514/44 R; 435/32; 435/6.12; 435/320.1; 435/356; 435/69.4

(57) ABSTRACT

Insulin gene therapy is one of many envisioned alternative treatments of diabetes. Diabetes gene therapy would be possible if insulin could be produced in a regulated and specifically in a sensitive manner dependent on the blood glucose level. Therefore, the present invention relates to a method for the isolation of GLP-1 expressing cells, to nucleic acids sequence construction or vectors useful for isolating GLP-1 expressing cell and to the GLP-1 expressing cells isolated therewith. Furthermore, the invention relates to a method of nucleic acids sequence construction or vectors under the control of the GLP-1 promoter expressing insulin in a recombinant GLP-1 expressing cell line. The cells of the present invention are particular useful for the treatment of diabetes and may be used in a gene therapy approach to treat diabetes and other disorders related to the nutrient metabolism.

### FIELD OF THE INVENTION

Insulin gene therapy is one of many envisioned alternative treatments of diabetes. Diabetes gene therapy would be possible if insulin could be produced in a regulated and specifically in a sensitive manner dependent on the blood glucose level. Therefore, the present invention relates to a method for the isolation of GLP-1 expressing cells (L cells), to nucleic acids sequence construction or vectors useful for isolating GLP-1 expressing cell and to the GLP-1 expressing cells isolated therewith. Furthermore, the invention relates to a method of nucleic acids sequence construction or vectors under the control of the GLP-1 promoter expressing insulin in a recombinant GLP-1 expressing cell line. The cells of the present invention are particular useful for the treatment of diabetes and may be used in a gene therapy approach to treat diabetes and other disorders related to the nutrient metabolism.

### BACKGROUND OF THE INVENTION

Diabetes is in the top 10, and perhaps the top 5, of the most significant diseases in the developed world. For at least 20 years, diabetes rates in North America have been increasing substantially. In 2005 there were about 20.8 million people with diabetes in the United States alone. According to the American Diabetes Association, there are about 6.2 million people undiagnosed and about 41 million people that would be considered prediabetic (American Diabetes Association., 2006). As in Malaysia, prevalence of known diabetics accounted for 1.2 million from the population (Malaysian Diabetes Association., 2007).

Achieving normal or near-normal circulating glucose levels is the primary goal of diabetes therapy. For those with Type 1 diabetes (in most cases) and some Type 2 diabetes (in their progressing stage) who can no longer make insulin, insulin replacement therapy is essential for treatment. The current standard of diabetes care for Type 1 diabetics includes orally delivered drugs and subcutaneous insulin injections (Tanya et al., 2001; Fowler, 2008). Insulin was initially prepared by isolation from animal pancreatic tissue, but it was not effective solution because of immunogenicity of animal insulin. Now insulin is prepared through recombinant DNA techniques using microorganisms. Use of recombinant insulin has decreased the immunogenicity of animal insulin, but factors such as multiple daily subcutaneous injections especially in precise and fixed quantities, frequent glucose monitoring and dietary restrictions care tiresome causes a heavy burden on diabetic patients (many of whom are very young) and their families.

Ideal glucose levels are rarely attainable in patients requiring insulin injections and this could lead to complications and other disorders as side effects for instance renal failure, diabetic ulcers and adult blindness (Peeples et al., 2007) not to mention short term acute complication such as hypoglycemia, Diabetic ketoacidosis and Hyperosmolar non ketotic coma. Long-term and short term complications of diabetes can be prevented if glucose can be maintained at normal level at all time. According to the Juvenile Diabetes Foundation however; every patient spends about $500,000 on diabetes management and treatment of diabetes-related complications during their life. Thus, diabetes mellitus is an important public health issue in terms of disease incidence, morbidity and mortality, as well as financial impact (public and personal) (Tanya et al 2001; Johnson et al., 2008).

For quite sometimes, pancreas transplants studies have been aimed to cure insulin-dependent diabetes mellitus (IDDM). Therefore, islet replacement strategies have become increasingly attractive options for patients at risk for severe diabetic complications. A major limitations of this approach however are the small number of organs available for transplantation or islet isolation, the relative scarcity of organs donors and the risks of major surgery (which is even higher in diabetic patients), graft rejection and (if successful) subsequent requirement for immunosuppressive therapy (Halvorsen et al., 2001). Thus, an important next step in developing curative treatments for diabetes will be the generation of a source of glucose-responsive and insulin-secreting cells that can be used for beta cell replacement.

Gene therapy has been highlighted as the most promising technology of the 21st century. Previous attempts by researchers worldwide for insulin gene therapy have largely concentrated on the manipulation of liver cells (Ruian et al., 2003). Genetic engineering of ectopic insulin production and secretion in antilogous non beta-cells is tested in different tissues including liver, muscle, pituitary-hepatopoietic, stem cells, fibroblasts and exocrine glands of gastrointestinal tract (Halvorsen et al. 2001. Creusot et al. 2004).

Another approach in gene therapy was to express the insulin gene from a glucose-responsive promoter (Mitanchez et al., 1997). In previous study, insulin expression was considered by proopiomelanocontin (POMC) promoter into murine intermediate pituitary lobe cells (Lipes et al., 1996) and by SV40 early promoter into AtT20 cell line (derived from the mouse anterior pituitary) (Moore et al., 1983). The result from these studies showed that pituitary cells efficiently secrete fully processed, mature insulin via a regulated secretory pathway, similar to islet β cells. However, insulin secretion was not glucose-regulated. Transfection of the GLUT-2 glucose transporter gene into insulin expressing AtT20 cells did result in glucose-stimulated insulin secretion, but maximal insulin secretion occurred at subphysiological glucose concentrations, again incurring risk of hypoglycemia (Davies et al., 1998).

A more advanced strategy is to start with cells that already have a regulated secretory pathway amenable to insulin storage and secretion. Incretin hormones for instance Glucose-dependent insulinotropic peptide (GIP) and glucagons-like peptide-1 (GLP-1) which was produced by the enteroendocrine (EE) cells play important roles in regulating and integrating many aspects of gastrointestinal and animal physiology (Sjolund et al 1983). Since GIP is secreted by gut K-Cells with a temporal pattern and in response to similar nutrients as insulin secretion by islet β-cells, it has been proposed that engineering gut K-Cells to produce insulin is a potential gene therapy to treat diabetes. To begin to test this hypothesis, GIP-producing cell lines were established and engineered. This cell line expressed the human insulin gene that linked to the downstream of the GIP promoter (GIP/Ins cells). Like K Cells in vivo, GIP/Ins cells expressed both insulin and GIP in response to the GIP secretagogues arginine, bombesin, and protein hydrolysates (Cheung et al., 2000; Ramshur et al., 2002).

Glucagon-like peptides-1 (GLP-1) is a product of gut L-cells located in the distal small intestine and released in the circulation in response to the nutrient ingestion and plays multiple roles in metabolic homeostasis following nutrient absorption (Baggio et al., 20001. Glucose, protein hydrolysates, specific amino acids, and fat are the major nutrients that stimulate GLP-1 release. In addition, it has reported that murine L cells (GLUTag) that were transfected by recombinant insulin gene, efficiently expressed insulin protein (Bara et al. 2008). Therefore. L cells same as K cells are sensitive towards glucose level in intestine and are able to process proinsulin to mature insulin.

Diabetes mellitus is a syndrome characterized by abnormally high blood glucose (hyperglycemia) and a disordered metabolism. Additional symptoms of diabetes mellitus include excessive thirst, glycosuria, polyuria, lipidemia and hunger (Watkins et al., 2003). The two principal forms of diabetes mellitus are known as types 1 and 2; Insulin-dependent diabetes mellitus, IDDM (more commonly referred to as type 1 diabetes) is the result of autoimmune destruction of the β-cells of pancreas. Non-insulin-dependent diabetes mellitus, NIDDM (more commonly referred to as type 2 diabetes) can result from genetic defects that cause both insulin resistance and insulin deficiency (Crofford et al., 1995). So basically, disorder in the insulin function is the main cause of Diabetes Mellitus.

In mammals, insulin is synthesized in the pancreas within the beta cells (β-cells) of the islets of Langerhans. Insulin is a hormone that causing liver cells to uptake glucose and store it in the form of glycogen. In addition, adipose tissues and skeletal muscle are stimulated by insulin to utilize blood glucose and storage of triglyceride in adipose tissue. Moreover, insulin regulates the synthesis of many genes that affect on metabolic pathway. Therefore the major metabolic derangements which result from insulin deficiency in IDDM are impaired glucose, lipid and protein metabolism (Crofford et al., 1995; Dodson et al., 1998).

The major goal of therapeutic intervention in type 1 diabetes is to reduce circulating glucose levels, which can be accomplished through several approaches, aimed at diabetes treatment. Thus, the survival and quality of life of patients with type 1 diabetes is completely dependent on the fluctuations of their blood glucose levels (Peek et al., 2007).

Insulin gene therapy is one of alternative treatment of type 1 diabetes (IDDM). Engineering non-pancreatic cells to produce insulin in response to a glucose load can be a successful approach in the treatment of diabetes (IDDM). But previous attempts on the manipulation of different cells have failed, because those cells do not have the ability to store hormones (Ruian et al., 2003, Halvorsen et al., 2001). Furthermore, studies have shown that expression of insulin gene with other promoters displayed transcriptional repression whereby they are not able to quench insulin production or secretion rapidly enough, again increasing the risk of hypoglycemia (Mitanchez et al., 1997). To achieve the right approach of treatment, it needs special promoter that can direct the expression of insulin in temporary manner and also be sensitive to glucose level.

Enteroendocrine (EE) cells are a complex population of diffusely distributed hormone producing intestinal epithelial cells. These hormones play important roles in regulating and integrating many aspects of gastrointestinal and animal physiology (Mutoh et al., 2000). There are more than 30 peptides currently identified as being expressed within the digestive tract. Although EE cells represent less than 1% of the intestinal epithelial cells, they represent the largest endocrine organ in the body. The regulatory peptides synthesized by the gut include hormones, peptide neurotransmitters and growth factors (Schonhoff et al., 2004).

Glucose-dependent insulinotropic peptide (GIP) and glucagon-like peptide-1 (GLP-1) are two of many EE cell-derived hormones that constitute the class of molecules referred to as the incretins. Incretins are molecules associated with food intake-stimulation of insulin secretion from the pancreas. GIP and GLP-1 have significant effects on insulin secretion and glucose regulation (Deacon et al., 2005).

GLP-1 is derived from the product of the glucagon gene. This gene encodes a preprotein that is differentially cleaved dependent upon the tissue in which it is synthesized. In the gut, prohormone convertase enzyme leads to release of GLP-1. Upon nutrient ingestion, GLP-1 is secreted from intestinal enteroendocrine L-cells that are found predominantly in the ileum, colon, duodenum and jejunum. The primary physiological responses to GLP-1 are inhibition of glucagon secretion and inhibition of gastric acid secretion and gastric emptying. The latter effect will lead to increased satiety with reduced food intake along with a reduced desire to ingest food. The action of GLP-1 at the level of insulin and glucagon secretion results in significant reduction in circulating levels of glucose following nutrient intake. Other major responses to the actions of GLP-1 include pancreatic β-cell proliferation and expansion concommitant with a reduction of β-cell apoptosis (death) (Fehmann et al., 1995; Deacon et al., 2005).

GLP-1 hormones respond to changes in the concentrations of lumenal nutrients but are refractive to changes in the levels of nutrients in the blood (Fehmann et al. 1995). GLP-1 secretion is under nutritional, hormonal and neuronal control. It is released into the circulation immediately after ingestion of a meal. GLP-1 potentiates insulin secretion following binding to receptors on islet β-cells (Hansotia et al., 2005). Therefore these hormones express insulin similar to the normal physiological induction as the insulin produced by the healthy pancreas.

In view of the major drawbacks of the background art as cited above it was an object of the present invention to provide novel means for the treatment of diseases related to irregular glucose or insulin levels, in particular for the treatment of diabetes I or II, obesity or other disorders related to nutrient metabolism in a subject requiring such a treatment. In this regard the present invention intends in a first aspect to solve the problem of providing novel GLP-1 expressing cells from a heterogeneous population of cells (STC-1). In a second aspect, the invention intends to provide novel construct to express insulin endogenously (preferably in gut cell) mimicking the normal physiological induction of insulin secretion for gene therapy use that may constitute a new therapeutic route for tackling diseases such as diabetes.

### SUMMARY OF THE INVENTION

The above problem is solved in a first aspect by a method for the isolation of GLP-1 expressing cells, comprising the steps of

a. providing a nucleic acid construct comprising the GLP-1 promoter sequence operable linked to an antibiotic resistance marker gene,
b. introducing said nucleic acid construct into a population of cells suspected to contain GLP-1 expressing cells,
c. culturing the cells of b. in the presence of the antibiotic corresponding to the antibiotic resistance marker gene,
d. selecting a cell clone which shows resistance to the antibiotic, and
e. optionally, confirming the expression of GLP-1 in the selected cell clone.

In this study, the GLP-1 promoter was used to determine its efficacy in governing the expression of insulin in vivo and in vitro in a recombinant cell line model. In addition pure L cells were extracted from a heterogeneous population of STC-1 cells to provide means for an insulin gene therapy in the gut cells. Since GLP-1 is secreted from L-cells in a temporal pattern similar to insulin and also responds to nutrients comparable to insulin secretion by islet β-cells, it is proposed that engineering gut L Cells to produce insulin is a potential new route for gene therapy to treat diabetes. The present invention introduces a plasmid vector capable of expressing the insulin gene under the control of the GLP-1 promoter. This construct can be useful for the treatment of diabetes and other hyperglycemic disorders. The invention also provides an L cell line that is useful for studying intestinal cells physiology and activities.

The term “operable linked” describes in the context of the present disclosure that nucleic acid sequences which are intended to be “operable linked” are connected such that the functional features of their sequence perform their biological function when introduced into a cell. For the present case a promoter sequence is “operable linked” to a nucleic acid sequence, for example a gene sequence or in particular an open reading frame (ORF), when the promoter is sufficient to induces the expression of said gene sequence. This is the case if the promoter sequence is placed upstream of the five prime region of the gene that is intended to be expressed under the control of said promoter. The person of skill in the relevant art is well acquainted with the requirements of the expression of a gene and therefore can easily combine a promoter sequence with a target sequence in order to allow the targets sequence expression under the control of said promoter.

The expression vector containing a recombinant gene for a polypeptide constructs or a fusion protein construct, for example of insulin, allows the expression of the recombinant gene in gut cells. Such an expression vector incorporates the recombinant gene, preferably insulin (FIG. 15), and vector features such as the appropriate regulatory DNA sequences for transcription and translation, for phenotyping and to allow a temporal or other control of the expression. Further features may relate to RNA binding and post-expression manipulation of the expressed product.

For the present invention, the most important expect is the GLP1 promoter being used as a regulatory sequence that governs the expression of the recombinant gene—preferably human insulin in temporary manner.

The expression vector generally will include structural features such as a promoter (of GLP-1), an operator, a regulatory sequence and a transcription termination signal. The expression vector can be synthesized from any base vector that is compatible with the host cell or higher organism and will provide the foregoing features. The regulatory sequences of the expression vector will be specifically compatible or adapted in some fashion to be compatible with the eukaryotic host cells. Post-expression regulatory sequences, which cause secretion of the polypeptide construct, can be included in the eukaryotic expression vector. It is especially preferred that the expression vector exhibit a stimulatory effect upon the host cell such that the polypeptide construct is overproduced relative to the usual biosynthetic expression of the host.

In one embodiment of the herein described invention the GLP-1 expressing cells are L cells, in particular intestinal L cells. Further encompassed are preferred embodiments, wherein a population of cells suspected to contain GLP-1 expressing cells is derived from the mammalian intestine, preferably from an endocrine tumor of the intestine, most preferably the population is a heterogeneous population of STC-1 cells.

Another embodiment relates to a method according to the invention, wherein the antibiotic is selected from the group comprising zeocin or geneticin (neomycin). A variety of other selectable markers can be incorporated into the target cells of the invention. For example, a selectable marker which confers a selectable phenotype such as drug resistance, nutritional auxotrophy, resistance to a cytotoxic agent or expression of a surface protein, can be used. Selectable marker genes which can be used include neo, gpt, dhfr, ada, pac, hyg and hisD. The selectable phenotype conferred makes it possible to identify and isolate recipient target cells.

Further preferred is in another embodiment a method according to the invention, wherein the GLP-1 promoter is the rat GLP-1 promoter, preferably the promoter comprises the sequence according to FIG. 15.

In a second aspect the problem of the present invention is solved by a population of cells isolated by a method according to the herein above described inventive method.

In a third aspect the problem is solved by a nucleic acid comprising the sequence of the GLP-1 promoter operable linked to an antibiotic resistance marker gene, in particular zeocin or geneticin (neomycin). The person of skill in the relevant art is aware of further resistance marker genes which may be used in the context of the present invention. Therefore other antibiotics known in the art are encompassed by the present invention as well.

A next embodiment of the above third aspect of the invention relates to a nucleic acid comprising the sequence of the GLP-1 promoter operable linked to the insulin gene, in particular mammalian insulin, for example the human insulin gene according to the sequence of FIG. 16.

Yet another embodiment relates to a nucleic acid according to the invention, wherein the GLP-1 promoter is a sequence derived from a mammalian GLP-1 gene, such as a mouse, rat or human GLP-1 gene, for example a GLP-1 promoter comprising the sequence according to FIG. 15.

One embodiment is directed to a nucleic acid according to the invention, which is further comprising an antibiotic resistance marker gene.

In a fourth aspect the problem of the present invention is solved by an expression vector comprising the nucleic acid of the invention. Such an expression vector is preferably a mammalian expression vector, more preferably a human expression vector.

In a fifth aspect the inventive solution of the posed problem relates to a cell transformed with the nucleic acid according to the described invention or an expression vector as described herein above. According to the invention, the cell is a mammalian cell, preferably a mouse, rat or human cell, for example the cell is derived from the gut and is preferably an intestine L cell.

A sixth aspect solves the above problem by a method for the expression of insulin in a cell, comprising the steps of

a. providing a nucleic acid construct comprising the sequence of the GLP-1 promoter operable linked to the sequence of the insulin gene,
b. introducing said nucleic acid construct into a target cell.

By the above method insulin, preferably human insulin, is expressed under the control of the GLP-1 promoter. Full length Insulin is preferred for the purpose of the invention. Therefore, by the above method, target cells can produce insulin, or functional equivalents thereof, upon the natural stimuli of the GLP-1 system.

In a further embodiment the method for the expression of insulin in a cell according to the invention is preferred, wherein the target cell is a GLP-1 expressing cell, preferably a cell derived from the intestine, more preferably an intestine L cell, most preferably said target cell is a cell isolated by a method according to the above described embodiments of the invention.

Cells to be transfected in order to produce insulin can be obtained from gut cells. For example, primary and secondary cells which can be transfected by the present method. In particular preferred for the invention are L cells of the gut.

Encompassed by the embodiments of the present invention are further the above disclosed methods which are preferably in-vitro or ex-vivo methods. In one embodiment the cells of the invention are not human embryonic stem cells.

In a seventh aspect the invention relates to a method of producing an insulin expressing cell, wherein the method comprises the steps of method for the expression of insulin in a cell as described herein above, and a cell produced therewith.

In an eighth aspect the invention relates to a method of treatment of a subject suffering from a disease related to the blood insulin level or blood glucose level, the method comprising administering to a patient a therapeutically effective amount of a nucleic acid or an expression vector or a cell according to the embodiments of the herein described invention.

In another embodiment the treatment according to the invention is directed to a disease related to the blood insulin level or blood glucose level, such as classical hyperglycemia, wherein the disease is selected from the group consisting of diabetes I, diabetes II, and/or obesity.

A variety of modes of administration are effective in systemic treatment, such as injection, including intravenous, intramuscular, subcutaneous, and intraperitoneal injection; transmembrane or transdermal administration, using suitable suppositories or sprays; and, if properly formulated, oral administration. Suitable excipients for injection include various physiological buffers, such as Hank's solution and Ringer's solution; suitable transmembrane or transdermal formulations contain penetrants such as bile salts or fusidates; and typical oral formulations contain protective agents which inhibit the digestion of the active ingredient. Also available are various slow-release formulations involving macromolecular matrices such as pyrrolidones and methylcellulose. Alternate drug delivery systems include nanoliposomes, chitosan and other nanocarriers

The herein described embodiments of the invention are in particular useful for a gene therapy for the treatment of diabetes. The inventive methods, nucleic acids, vectors and cells may be used in order to provide a patient with cells expressing insulin under the control of the GLP-1 promoter.

While the present invention has been described with specificity in accordance with certain of its preferred embodiments, the following examples serve only to illustrate the invention and are not intended to limit the invention within the principles and scope of the broadest interpretations and equivalent configurations thereof.

### DESCRIPTION OF THE DRAWINGS

This invention will be described and understood when read with reference to the accompanying drawings, in which:

FIG. 1: shows the Glu.BS plasmid map and the restriction sites position.

FIG. 2: shows the pJET1.2 cloning vector map and the multiple cloning sites

FIG. 3: shows the Ins/pJET and the GLP-1pro/pJET plasmid map and the position of restriction sites

FIG. 4: shows the features of pBudCE4.1 vector and the restriction sites position

FIG. 5: shows the features of pBud (promoter EF less) vector and the restriction sites position

FIG. 6: shows Ins/pbud (A) and GLP-1/Ins/pbud (B) plasmid map and the position of restriction sites

FIG. 7: shows the positions of primers used for sequencing of GLP-1/Ins/pbud plasmid

FIG. 8: shows Neo/pJET (A) and Neo/pBlu (B) plasmid map and the position of restriction sites

FIG. 9: shows pBluescript II SK (+) cloning vector map and the multiple cloning sites sequences

FIG. 10: shows GLP-1-Ex/pJET (A) and GLP-1/Neo/pBlu plasmid map and the position of restriction sites

FIG. 11: shows the positions of primers used for sequencing of GLP-1/Neo/pbud plasmid

FIG. 12: shows the result of RT-PCR for confirmation of extracted L cell line. 5 L cell clones that were transfected by GLP-1/Neo/pBlu plasmid were considered for RT-PCR analysis. Lines 2-6 are the results of RT-PCR by β-actin primers and lines 8-11 are the results of RT-PCR by GLP-1 primers. The first line is 100 bp DNA ladder.

FIG. 13: shows the result of immunohistochemistry analysis. The arrows indicate the L cells that express human insulin. The left-up picture is the cells that were observed by DAPI filter (that indicate nucleus of the cells and right-up picture is the same position with FITC filter. The down picture is the combine of two previous pictures (DAPI and FITC filter).

FIG. 14: shows the result of immunohistochemistry analysis. The arrows indicate the L cells that express human insulin. The left-up picture is the cells that were observed by DAPI filter (that indicate nucleus of the cells and right-up picture is the same position with FITC filter. The down picture is the combine of two previous pictures (DAPI and FITC filter).

FIG. 15: shows the Sequence of Rat GLP-1 Promoter from gene bank (ref|NW_{—}047655.1; SEQ ID No: 1). The primers forward and reverse that were used for PCR amplification are highlight.

FIG. 16: shows the Sequence of Human Insulin gene from gene bank (ref|NG_{—}007114.1; SEQ ID No: 2). The primers forward and reverse that were used for PCR amplification are highlight

FIG. 17: shows the sequence of Neomycin resistant Gene from pcDNA3 vector (SEQ ID No: 3.) The primers forward and reverse that were used for PCR amplification are highlight

### DETAILED DESCRIPTION OF THE INVENTION

### GLP-1 Promoter

The GLP-1 promoter (glucagon) was obtained from the rat genomic sub clone Glu.BS plasmid containing the glucagon promoter (−2300 bp), the first exon and 100 bp of first intron of the rat glucagon gene in the pBS-SK+ (pBluscript phagemid vector) (FIG. 1). The Glu.BS plasmid was used as a source for the GLP-1 promoter sequence (Gosmain et al., 2007).

Previous studies demonstrated that ˜2300 bp fragment of rat proglucagon sequence is essential for the expression of GLP-1 gene in intestinal L cell (Jin et al., 1995). The sequence of rat glucagon was checked on gene bank (ref|NW_{—}047655.1) (Appendix 1). A fragment of rat proglucagon gene (pro Glu) was amplified from Glu.BS plasmid by PCR. Table 1 shows the sequences of primers and the position of restriction sites. Spe I (−2214) and Hind III (+77) sites were included on the upstream and downstream primers, respectively to facilitate subsequent cloning.

TABLE 1
Sequences and the position of restriction site
of GLP-1 primers
Primers

| | |
|---|---|
| ProM-F | 5′ AT GAG AAA GCT TGT AGA CAG GTG GAG 3′ |
| | Hind III |
| ProM-R | 5′ AC AAC ACT AGT GCT TCC AGT CAA ACC 3′ |
| | Spe I |

### The Insulin Gene

The human insulin gene was obtained from a human genomic DNA. The genomic DNA was extracted from human blood by manual method. The sequence of human insulin was checked on gene bank (ref|NG_{—}007114.1) (Appendix 2). Based on previous studies, about 1800 bp of insulin gene constitutes of introns, exons and other fragments that are needed for insulin expression.

The fragment of human insulin gene was amplified by PCR from human genomic DNA. The sequences of primers and the position of restriction site are showed in the table 2. The Sal I (+18) and BamH I (+1844) restriction sites were designed upstream and downstream of primers to facilitate subsequent cloning.

TABLE 2
Sequence of forward and reverse primers to
amplify insulin gene
Primers

| | |
|---|---|
| InsCo-F | 5′ AA GTT GTC GAC AGG CTG CAT CAG AAG 3′ |
| | Sal I |
| InsCo-R | 5′ A TAG GAT CCA CAG GGA CTC CAT CAG 3′ |
| | Bam H I |

### Purification of PCR Products

Following amplification, PCR products (GLP-1 promoter and insulin gene) were purified from agarose gel to omit undesired bands, primer dimmers and leftover of PCR mixture by DNA Gel Extraction kit.

Ligation with pJET1.2 Cloning Vector

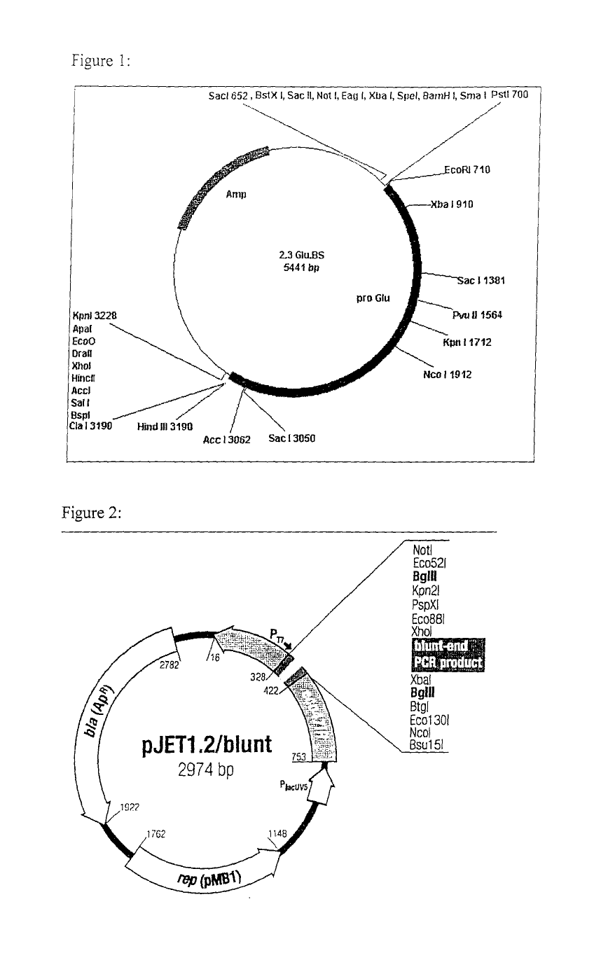

Pure PCR products (GLP-1 promoter and Insulin gene) were sub-cloned into the pJET1.2 cloning vector. The pJET1.2 cloning vector is an advanced positive selective system for the highest efficiency cloning of PCR products. Additionally, this system increases the effectiveness of restriction enzyme activity by creating enough space to be placed on the restriction sites. Moreover, sequencing of PCR products are more convenient in the plasmid form. This vector contains a lethal gene, which is disrupted by ligation of a DNA insert into the cloning site. As a result, only cells with recombinant plasmids are able to propagate (FIG. 2). The recombinant plasmids are named GLP-1pro/pJET (GLP-1 promoter inside the pJET1.2 cloning vector) and Ins/pJET (Insulin gene inside the pJET1.2 cloning vector) (FIG. 3).

Transformation into TOP-10

The ligation products (GLP-1pro/pJET and Ins/pJET) were transformed into the bacteria competent cells by head shock method to amplify plasmids construct (FIG. 3). The E. coli strain TOP-10 was employed as bacterial host for propagation of plasmid in whole project. Competent bacterial cells were prepared by treating the cell with a divalent cation like calcium chloride. The pJET1.2 cloning vector includes Ampicillin selectable marker (antibiotic resistance markers) that allows only cells that receive recombinant vector, grow in the selective medium. Nevertheless, these selection steps did not absolutely guarantee that the DNA insert was present in the cells. Further investigations of the resulting colonies were performed to confirm that cloning was successful. These were accomplished by means of restriction mapping analysis and DNA sequencing.

### Plasmid Extraction

Some single colonies randomly chose and were cultured on the selective medium to grow overnight. Recombinant plasmids were isolated from the bacterial by plasmid miniprep kit for further analysis. The size of GLP-1pro/pJET is about 5265 bp and Ins/pJET is about 4800 bp (FIG. 3).

### Restriction Mapping Analysis

Ins/pJET plasmid were digested by Sal I and BamH I restriction enzymes and GLP-1 pro/pJET plasmid were cut by Spe I and Hind III restriction enzymes to examine the correctness of the plasmid structure. Consequence of Ins/pJET plasmid digestion with Sal I and BamH I, were two fragments, insulin gene with the size of 1826 bp (insert) and linear pJET1.2 cloning vector with the size of 2974 bp (vector). In addition, consequence of GLP-1 pro/pJET plasmid digestion with Spe I and Hind III were two fragments, GLP-1 promoter with the size of 2291 bp (insert) and linear pJET1.2 cloning vector with the size of 2974 bp (vector). Only the colonies that produce these fragments during digestion analysis were selected for next experiments.

### Sequencing

Random colony samples which have gone through extraction of Ins/pJET and GLP-1 pro/pJET plasmid were sent for sequencing analysis to confirm the correctness of nucleotides sequence of insulin gene and GLP-1 promoter. The results of sequencing were compared with sequence of rat GLP-1 promoter and human insulin gene in gene bank database (ref|NW_{—}047655.1 and ref|NG_{—}007114.1) (Appendix 1, 2).

### Construct GLP-1/Ins/pbud Plasmid

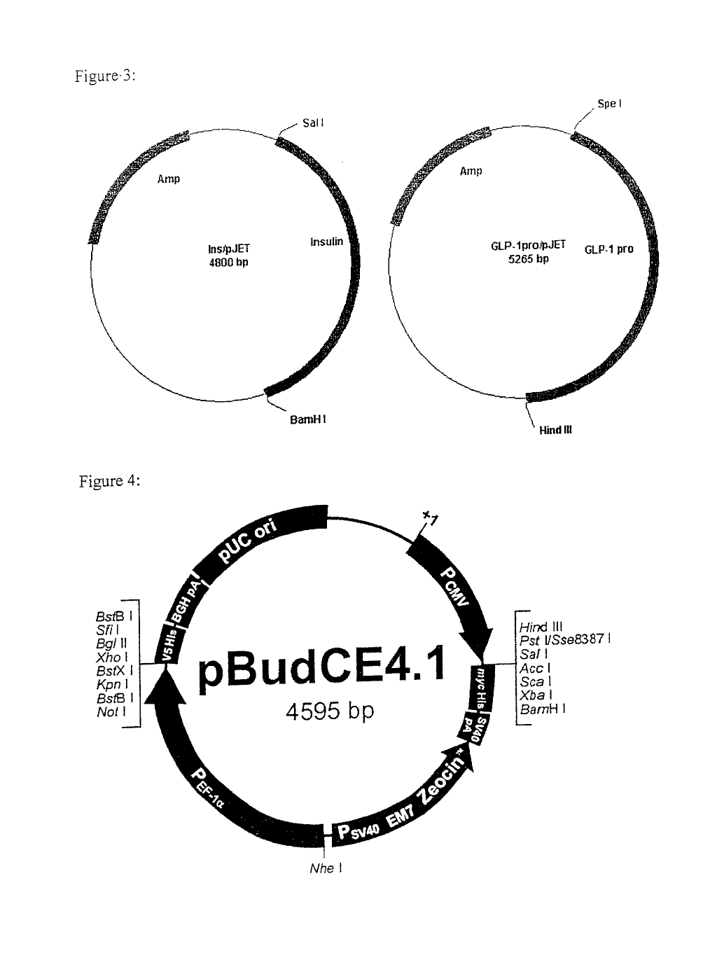

To construct GLP-1/Ins/pbud plasmid, the pBudCE4.1 was employed as cloning vector. The pBudCE4.1 vector was designed for simultaneous expression of two genes in mammalian cell line. The vector contains the two promoters (CMV and EF-1α promoter) and two multiple cloning sites that allow independent expression of two recombinant proteins. The pBudCE4.1 includes Zeocin resistant gene for selection in E. coli as well as serves to create stable mammalian cell line. Most E. coli strains are suitable for the growth of this vector including TOP-10 and DH5α (FIG. 4).

It should be noted that, CMV promoter and EF-1α promoter was eliminated in the new construct development, because the aim of the project is to study of GLP-1 promoter ability to express insulin gene, so to avoid complication and confusion with the GLP-1 promoter, promoters of the vector were deleted. Therefore, EF-1α promoter was omitted completely and CMV promoter was replaced with GLP-1 promoter.

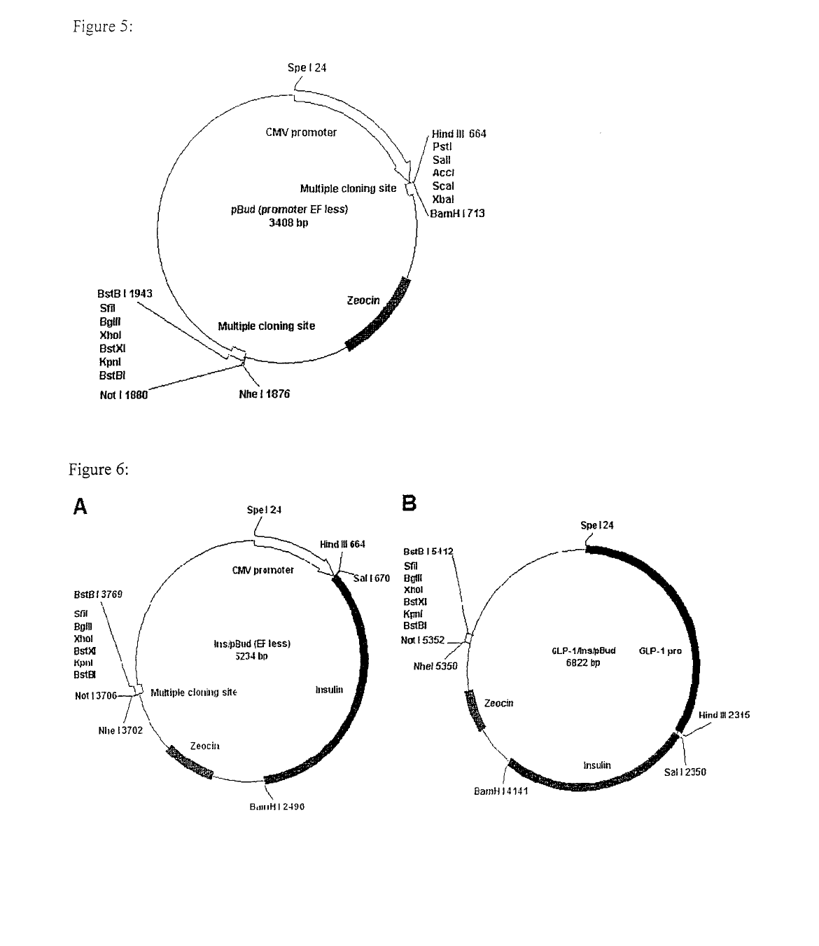

In order to omit EF-1α promoter, the pBudCE4.1 vector was digested with Nhe I and Not I restriction enzymes. Next, pBud vector band was purified from agarose gel to omit undesired bands (EF-1α promoter) as well as any leftover mixture of digestion by DNA Gel Extraction kit. The pBud vector (“pBud pro EF less”) which now has lost its EF-1α promoter has two different sticky ends that are not able to match with each other because it was digested by two different restriction enzymes. In order to construct the circle vector, the “pBud pro EF less” fragment was treated by Klenow Fragment enzyme to make blunt ends. The blunt ends facilitate subsequence ligation in order to recircle the vector (FIG. 5).

The treated fragment was ligated by T4 DNA ligase enzyme to attach the two blunt ends with each other and make circle “pBud pro EF less” vector (FIG. 5). This new vector was employed in producing GLP-1/Ins/pbud plasmid.

### Construct GLP-1/Ins/pbud Plasmid

The insulin gene and GLP-1 promoter were inserted into the “pBud pro EF less” vector in two steps. At first, the Ins/pJET plasmid (containing Human Insulin gene) and “pBud pro EF less” vector were digested by suitable restriction enzymes (Sal I and BamH I) to create insulin gene (insert) and linear pBud vector with same sticky ends. These digested fragments were purified from gel electrophoresis by Gel DNA Recovery Kit to omit undesired fragments. Insert (insulin) and vector (pBud pro EF less) were ligated to construct Ins/pbud plasmid include insulin gene in the Sal I and BamH I site (FIG. 6A). The ligation product was transformed into the E. coli strain TOP-10 as host bacterial for propagation of plasmid.

Single colonies obtained from Ins/pbud plasmid transformation process were extracted to check the correctness of plasmid content. In this order, some single colonies were randomly selected to extract their plasmid. The plasmids were digested by Sal I and BamH I restriction enzymes. The plasmids that contain insulin gene had two fragments on the gel that were the same size in compare with the insert (insulin gene 1826 bp) and vector (pBud pro EF less vector 3400 bp).

In the second step, GLP-1 promoter was inserted to the Ins/pbud plasmid in such a manner that it was placed upstream of the insulin gene (FIG. 6B). In this case, the GLP-1pro/pJET plasmid (containing rat GLP-1 promoter, FIG. 3) and Ins/pbud were digested with Spe I and Hind III restriction enzymes to generate GLP-1 promoter fragment (as insert) and linear Ins/pbud fragment (as vector) with sticky ends. These digested fragments were purified from gel electrophoresis by Gel DNA Recovery Kit to omit undesired fragments. Insert (GLP-1 promoter) and vector (Ins/pbud plasmid) were ligated to construct GLP-1/Ins/pbud plasmid include GLP-1 promoter in the Spe I and Hind III sites and insulin gene in the Sal I and BamH I sites (FIG. 6B). The ligation product was transformed into the E. coli strain TOP-10 as host bacteria for propagation of plasmid.

The accomplishment of GLP-1/Ins/pbud plasmid transformation was examined by analyzing several single colonies. In this order, some colonies randomly were selected to extract their plasmid. The plasmids were digested by Spe I and Hind III restriction enzymes. The correct plasmids have two fragments on the gel that were the same size in compare with the insert (GLP-1 promoter 2291 bp) and vector (Ins/pbud plasmid 4790 bp).

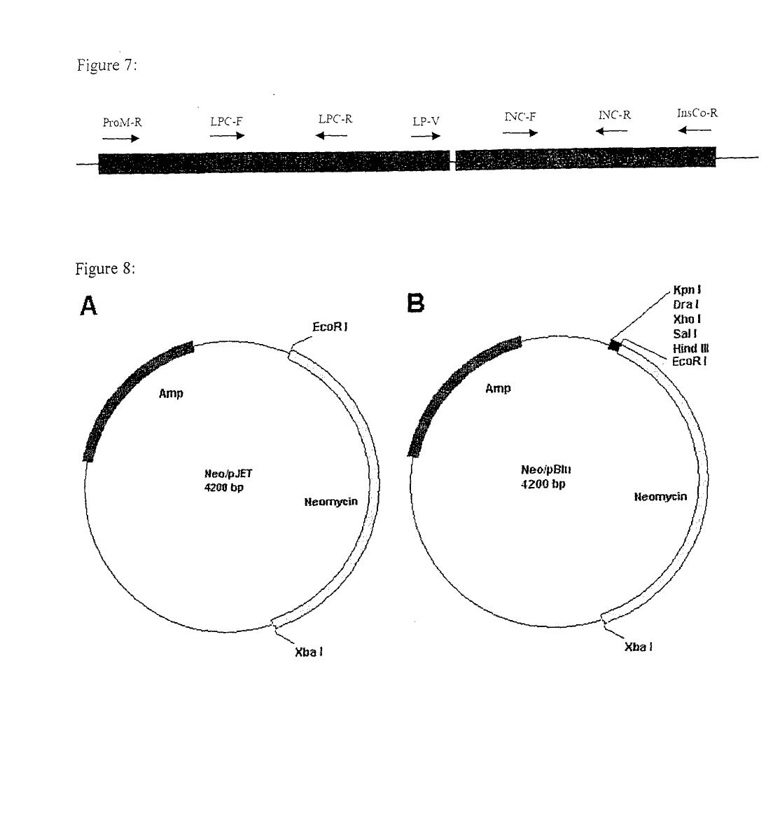

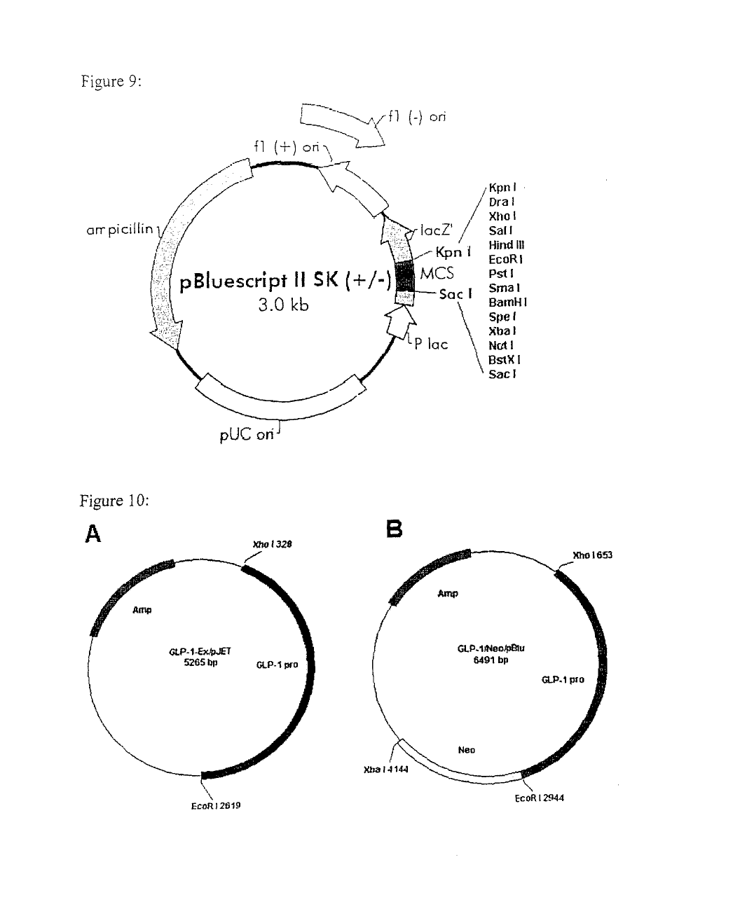

One sample from extraction of GLP-1/Ins/pbud plasmid was sent for sequencing analysis to confirm the correctness of nucleotides sequence of insulin gene and GLP-1 promoter. The positions of primers that used for sequencing of GLP-1/Ins/pbud are showed in FIG. 7 and the sequences of primers are listed in table 3. The results of sequencing were compared with sequence of rat GLP-1 promoter and human insulin gene in gene bank database (ref|NW_{—}047655.1 and ref|NG_{—}007114.1) (Appendix 1, 2).

TABLE 3
The sequence of primers that used for sequencing
of GLP-1/Ins/pbud plasmid
Primers for sequencing of GLP-1/Ins/pbud

| | |
|---|---|
| ProM-R | 5′ AC AAC ACT AGT GCT TCC AGT CAA ACC 3′ |
| LP-V | 5′ G ACG TCA AAA TTC ACT TCA GAG AGC 3′ |
| LPC-F | 5′ G CTA AAT CTG GGT GTC CAA GTG 3′ |
| LPC-R | 5′ A AGC TCC ATG TCC ACC AGT TAG 3′ |
| InsCo-R | 5′ A TAG GAT CCA CAG GGA CTC CAT CAG 3′ |
| INC-F | 5′ CT CAC GGC AGC TCC ATA GTC 3′ |
| INC-R | 5′ TGT TCC ACA ATG CCA CGC TTC 3′ |

### Construct Plasmid for L Cell Selection

### Neomycin Gene

Suitable selected marker for mammalian cell line is needed to be expressed under GLP-1 promoter to extract L cells from heterogeneous population of STC-1 cell line. In this order, neomycin resistant gene causing resistance against geneticin antibiotic in mammalian cell line was placed downstream of the GLP-1 promoter in the new constructs. After transfection of the STC-1 cell line with plasmid containing neomycin resistant GLP-1 promoter, the cells only could determine GLP-1 promoter (L cell respectively) and express neomycin resistant protein were able to survive under geneticin antibiotic treatment condition.

The neomycin resistant gene was amplified from pcDNA3 plasmid by PCR with two specific primers that include restriction enzyme sites (EcoR I and Xba I respectively) to facilitate subsequent cloning (Table 4) (Appendix 3). The PCR product with 1202 bp fragment was purified from agarose gel to omit undesired bands, primer dimmers and leftover PCR mixture. Pure PCR product was sub-cloned into the pJET1.2 cloning vector to construct Neo/pJET plasmid (FIG. 8A). The ligation product was transformed into the E. coli strain TOP-10 competent cells to amplify new construct.

TABLE 4
Sequence of forward and reverse primers to amplify
neomycin resistant gene
Primers

| | |
|---|---|
| NEc-F | 5′ GA ATT CCA GAA GTA GTG AGG AGG 3′ |
| | EcoR I |
| NXb-R | 5′ T CTA GAT ACA TTG ATG AGT TTG GAC 3′ |
| | Xba I |

The Neo/pJET plasmid was digested by EcoR I and Xba I restriction enzymes. Consequence of Neo/pJET plasmid digestion was neomycin resistant gene with size of 1202 bp (insert) and pJET1.2 cloning vector with size of 2974 bp (vector). For confirmation, one sample from extraction of Neo/pJET plasmid was sent for sequencing analysis to check the correctness of nucleotides sequence of neomycin resistant gene. The result of sequencing was compared with sequence of neomycin resistant gene in pcDNA3 plasmid sequence (ACCESSION EF550208). The single colony that had correct structure and sequence was selected for next experiment.

Insertion of Neomycin Gene into the pBluescript Plasmid:

The pBluescript II phagemid (plasmid with a phage origin) is cloning vector designed to simplify commonly used cloning procedure. This vector has an extensive polylinker with unique restriction enzymes to facilitate insertion of new fragments (FIG. 8).

The neomycin resistant gene was inserted to the pBluescript plasmid in such a manner that it was placed between EcoR I and Xba I resistant sites (FIG. 8B). In this case, Neo/pJET plasmid and pBluescript vectors were digested with the same restriction enzymes, EcoR I and Xba I, to generate linear neomycin resistant gene fragment (as insert) and linear pBluescript vector with sticky ends. These digested fragments were purified from gel electrophoresis by Gel DNA Recovery Kit to omit undesired fragments. Insert (neomycin resistant gene) and vector (pBluescript plasmid) were ligated to construct Neo/pblu plasmid include neomycin resistant gene in the EcoR I and Xba I sites (FIG. 8B). The ligation product was transformed into the E. coli strain TOP-10 as host bacteria for propagation of plasmid.

The accomplishment of Neo/pblu plasmid transformation was again examined by analysing several single colonies. In this order, some colonies randomly were selected to extract plasmid. The plasmids were digested by EcoR I and Xba I restriction enzymes. The correct plasmids had two fragments on the gel that were the same size in compare with the insert (Neomycin resistant gene 12002 bp) and vector (pBluescript plasmid 3000 bp).

One sample from extraction of Neo/pblu plasmid was sent for sequencing analysis to confirm the correctness of nucleotides sequence of neomycin resistant gene. The results of sequencing were compared with neomycin resistant gene in gene bank database (ACCESSION EF550208).

PCR GLP-1 with New Primers

To construct the GLP-1/Neo/pblu plasmid, GLP-1 promoter was placed upstream of neomycin gene in the EcoR I and Xho I restriction sites. In this order, GLP-1 was amplified with other primers include EcoR I and Xho I restriction enzyme sites. Sequences of forward and reverse primers to amplify GLP-1 promoter are showed in the table 5. Primers include EcoR I and Xho I restriction enzyme sites.

TABLE 5
Sequence of forward and reverse primers to
amplify GLP-1 gene
Primers

| | |
|---|---|
| LP-F | 5′ G AAT TCG AGC TGA GAG GAG GTG TAG 3′ |
| | EcoRI |
| LP-R | 5′ C TCG AGA TAC CTG CCT ACC ACT GTC 3′ |
| | XhoI |

The GLP-1 fragment was purified from the agarose gel by the Gel DNA Recovery Kit, and then was sub-cloned into the pJET1.2 cloning vector to construct GLP-1-Ex/pJET plasmid (FIG. 10A).

### Construct GLP-1/Neo/pblu Plasmid:

The GLP-1 promoter was inserted to the Neo/pblu plasmid to produce GLP-1/Neo/pBlu plasmid. At first, the GLP-1 EX/pJET plasmid (FIG. 10A) and Neo/pblu plasmid (FIG. 8B) were digested by suitable restriction enzymes (Xho I and EcoR I) to create GLP-1 promoter fragment (insert) and linear Neo/pBlu vector with same sticky ends. These digested fragments were purified from gel electrophoresis by Gel DNA Recovery Kit to omit undesired fragments.

Next, Insert (GLP-1 promoter) and vector (Neo/pBlu plasmid) were ligated to construct GLP-1/Neo/pBlu plasmid include GLP-1 promoter in the Xho I and Ecor I sites and neomycin gene in the downstream of GLP-1 promoter in the position of EcoR I and Xba I sites (FIG. 10B). The ligation product was transformed into the E. coli strain TOP-10 as host bacterial for propagation of plasmid. The correctness of plasmid structure was considered by restriction enzyme mapping and sequencing. The positions of primers that used for sequencing of GLP-1/Neo/pbud are showed in FIG. 11 and the sequences of primers are listed in table 6 (Appendix 3).

TABLE 6
The sequence of primers that used for sequencing
of GLP-1/Neo/pbud plasmid
Primers for sequencing of GLP-1/Neo/pbud

| | |
|---|---|
| ProM-R | 5′ AC AAC ACT AGT GCT TCC AGT CAA ACC 3′ |
| LP-V | 5′ G ACG TCA AAA TTC ACT TCA GAG AGC 3′ |
| LPC-F | 5′ G CTA AAT CTG GGT GTC CAA GTG 3′ |
| LPC-R | 5′ A AGC TCC ATG TCC ACC AGT TAG 3′ |
| NXb-R | 5′ T CTA GAT ACA TTG ATG AGT TTG GAC 3′ |

### In Vitro Study

STC-1 cell line was derived from an endocrine tumor of the intestine (Rindi et al., 1990). It has been demonstrated that ˜7% and 5% of this heterogeneous population of cells produce immunoreactive glucose dependent insulinotropic polypeptide (GIP) and glucagon like polypeptide I (GLP-1), respectively. In addition, there was no immunoreactivity detected for insulin antibodies in STC-1 cell line (Rindi et al., 1990). Since, STC-1 cell line is suitable source of L cells; it was applied for in vitro studies.

The concentration of 5×104 cells/ml is proper for primary culture. Based on previous studies, STC-1 cells were grown in Dulbecco's modified Eagle's medium supplemented with 10% fetal bovine serum under an atmosphere 5% CO2 and 37° C. (Rindi et al., 1990). The media of culture was changed in regular interval. Then, the cells were passaged in the new flasks.

### MTT Assay

For assessment of antibiotic cytotoxicity, a common methodology is the MTT assay which has been widely used as a colorimetric approach based on the activity of living cells. MTT assay is a standard assay (an assay which measures changes in color) for measuring cellular proliferation. Yellow MTT (3-(4,5-Dimethylthiazol-2-yl)-2,5-diphenyltetrazolium bromide, a tetrazole) is reduced to purple formazan in the mitochondria of living cells. The absorbance of this colored solution can be quantified by measuring at a certain wavelength (usually between 500 and 600 nm) by a spectrophotometer.

The pBudCE4.1 and pBluescript plasmids were employed for expression of insulin gene and neomycin gene, include zeocin and geneticin (neomycin) resistant gene respectively.

Therefore, the MTT assay was done for both antibiotics to determine the appropriate concentration of the antibiotic that kills the entire STC-1 cells lacking the antibiotic resistant gene. In this case, STC-1 cells (without any antibiotic resistant gene) were treated with different concentration of zeocin and geneticin antibiotic. The concentration of antibiotics in the culture media was in the range of 0 to 1 mg/ml in 12 wells (Table 7).

TABLE 7
Concentration of zeocin and ampicilin antibiotic in the culture media

| | Con. Ant | | | | | | | | | | | |
|---|---|---|---|---|---|---|---|---|---|---|---|---|
| | ug/ml | | | | | | | | | | | |
| | 0 | 50 | 100 | 200 | 300 | 400 | 500 | 600 | 700 | 800 | 900 | 1000 |
| DMEM | 180 | 179.5 | 179 | 178 | 177 | 176 | 175 | 174 | 173 | 172 | 171 | 170 |
| Serum | 20 | 20 | 20 | 20 | 20 | 20 | 20 | 20 | 20 | 20 | 20 | 20 |
| Anti | 0 | 0.5 | 1 | 2 | 3 | 4 | 5 | 6 | 7 | 8 | 9 | 10 |
| Total | 200 | 200 | 200 | 200 | 200 | 200 | 200 | 200 | 200 | 200 | 200 | 200 |

Optical density of solutions was read at 560 nm on an ELISA plate reader. The absorbance of colored solution is directly proportional to the number of cells. Based on MTT assay result, the concentration of geneticin and zeocin antibiotic that are able to kill all the STC-1 cells (without antibiotic resistant gene) were 400 ug/ml and 500 ug/ml.

Transfection of pGLP-1/Neo/pBlu Plasmid

The L cell line was isolated from heterogeneous population of STC-1 cell line by pGLP-1/Neo/pBlu plasmids. This plasmid is able to express neomycin resistant gene under control of GLP-1 promoter. So, recombinant constructed plasmid (pGLP-1/Neo/pBlu plasmids) was transfected to the STC-1 cell line by transfection reagent (Lipofectamine), according to manufacturer's protocol. Selection of stable clones was performed by replacing medium the day after transfection with complete medium, supplemented with proper amount of G418 (Geneticin antibiotic) that measured in MTT assay (400 ug/ml). Medium was changed every 2-3 days, until individual clones of transfected cell appeared. Stable transfected cell clones were isolated for next step analysis.

### RT-PCR for Mouse GLP-1 Gene

Expression of mouse GLP-1 mRNA was detected by reverse transcription reaction by PCR to confirm the success of transformation work that has been carried out on the L cell line. GLP-1 protein is expressed cell specifically, so just L cells are able to produce GLP-1 mRNA. In this case, the result of RT-PCR approved the present of GLP-1 mRNA in the mouse L cell line that was extracted from STC-1 cell line.

Total RNA was extracted by using RNA Extraction Kit, according to manufacturer's protocol. Then, extracted RNA was digested with DNase I (free RNase). RT-PCR was carried out with total RNA according to proposed step in RT-PCR kit. The PCR reaction was carried out in a 30 ul final volume containing primers for control mRNA (mouse β-actin) and mouse GLP-1 mRNA. Primers were designed to amplify nucleotides 204-762 of coding sequence for mouse β-actin and 265-515 of the coding sequence for mouse glucagon (GLP-1) mRNA. Theses primers bind within two different exons, therefore, products generated from mRNA and genomic DNA can be easily distinguished. The upstream and downstream primers are used to amplify β-actin and GLP-1 mRNA are listed in table 8 and 9 respectively.

TABLE 8
Sequence of forward and reverse primers
for β-actin RT-PCR
Primers

| | |
|---|---|
| Ac-rt-F | 5′ GTG TGA TGG TGG GAA TGG GTC 3′ |
| Ac-rt-R | 5′ AG GAA GAG GAT GCG GCA GTG 3′ |

TABLE 9
Sequence of forward and reverse primers
for β-actin RT-PCR
Primers

| | |
|---|---|
| LP-rt-F | 5′ GGC ACA TTC ACC AGC GAC TAC 3′ |
| LP-rt-R | 5′ CA ATG GCG ACT TCT TCT GGG 3′ |

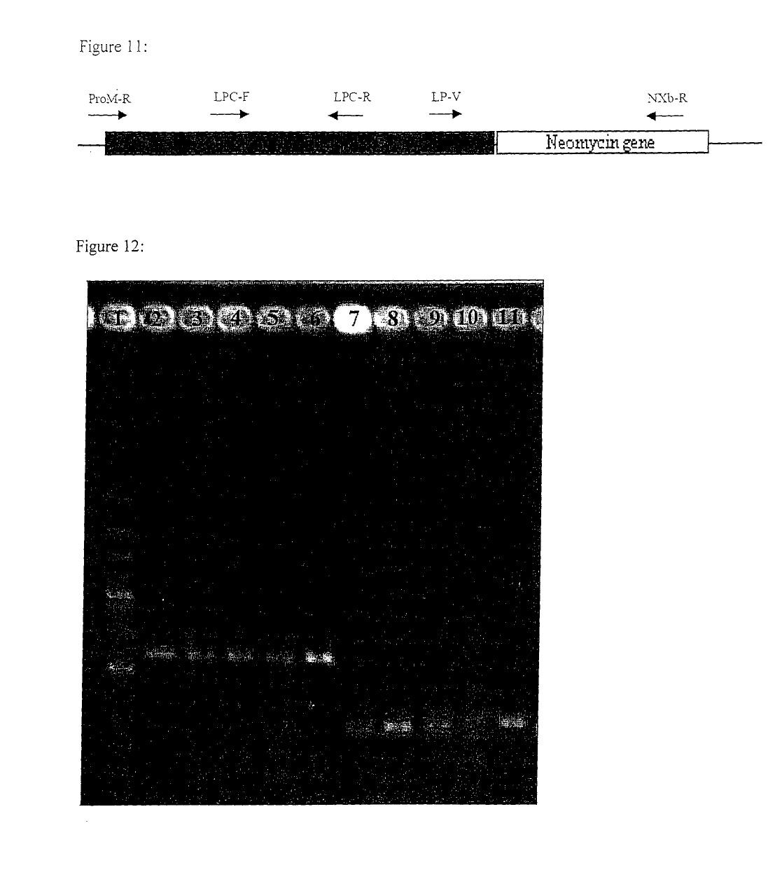

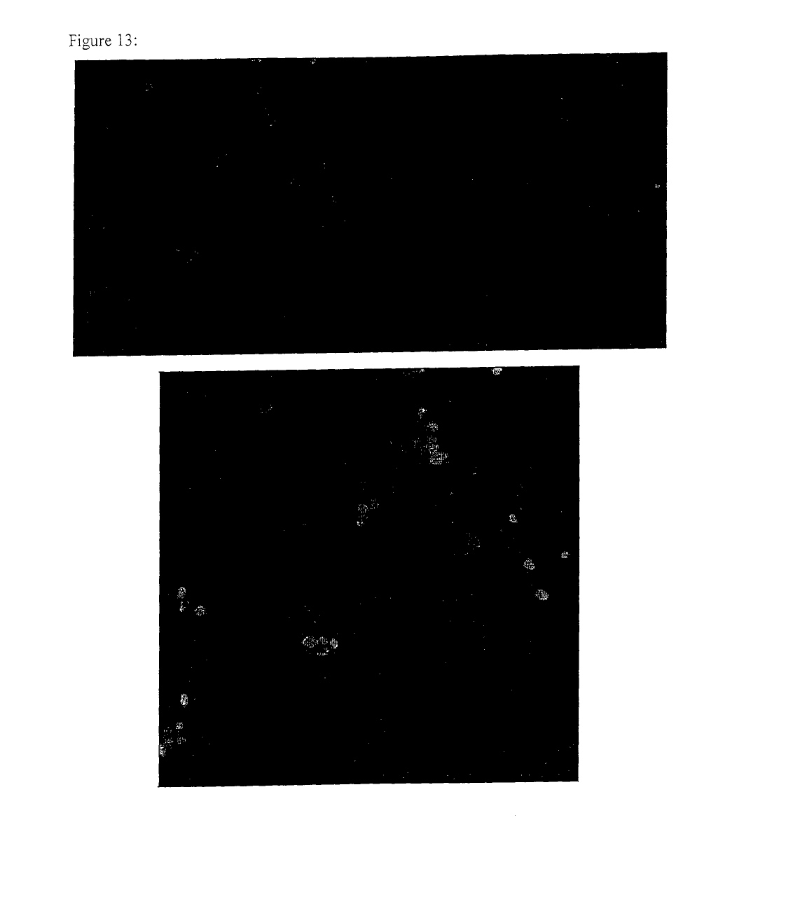

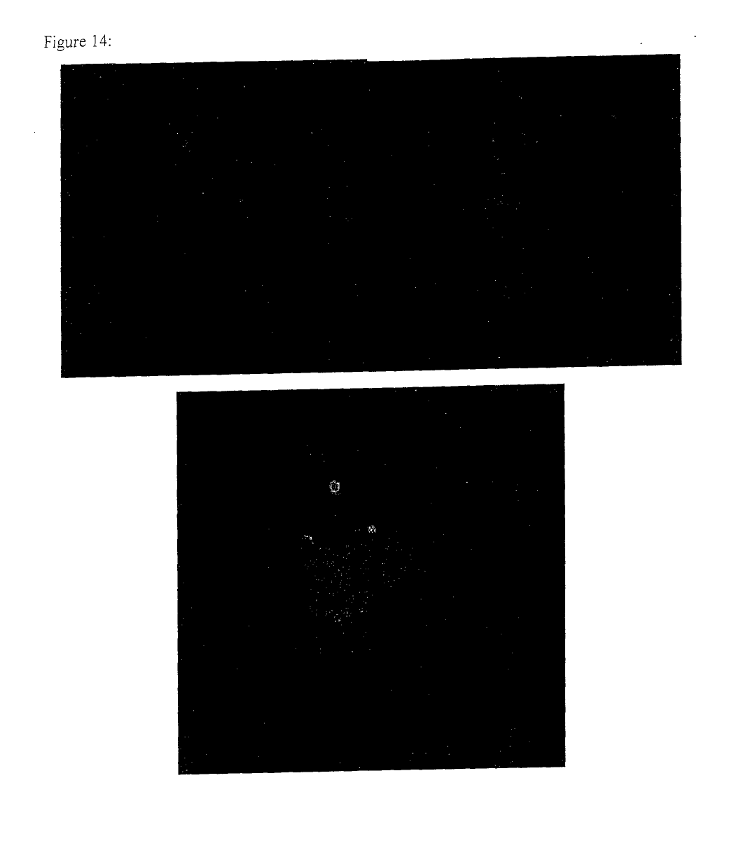

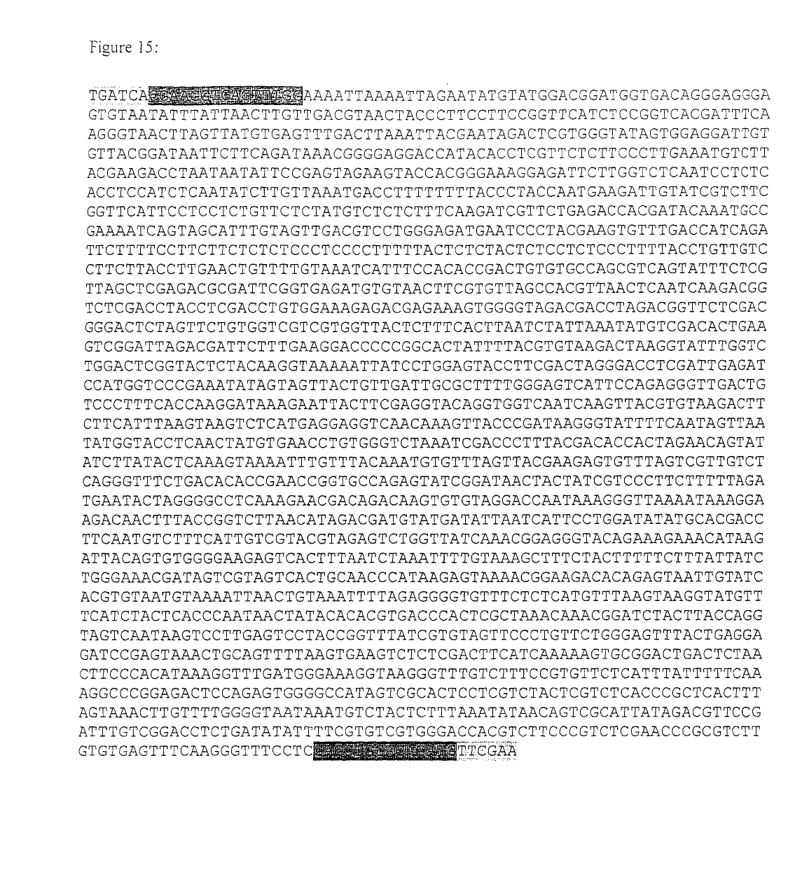

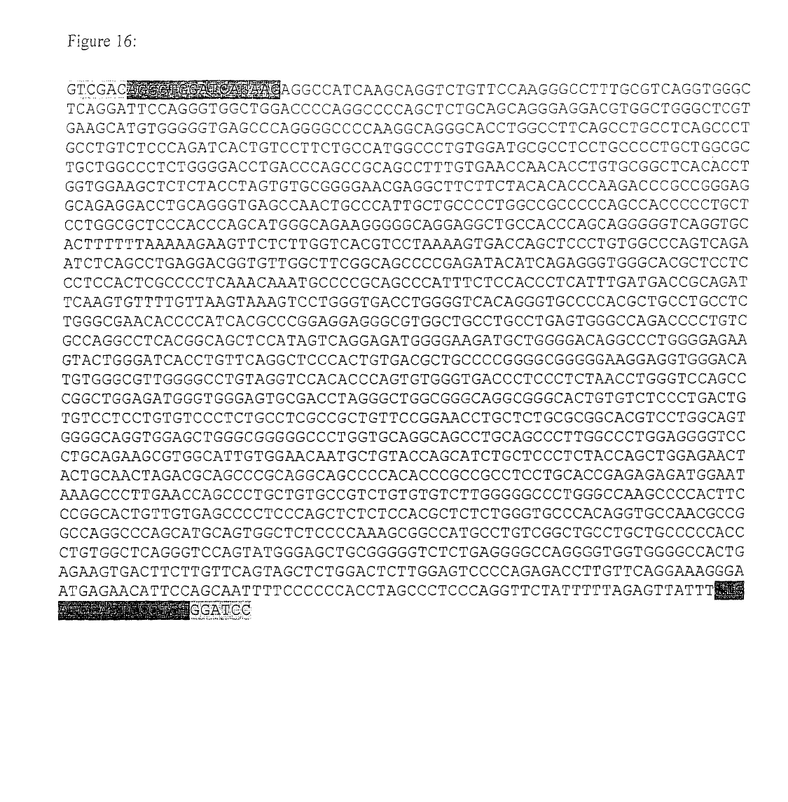

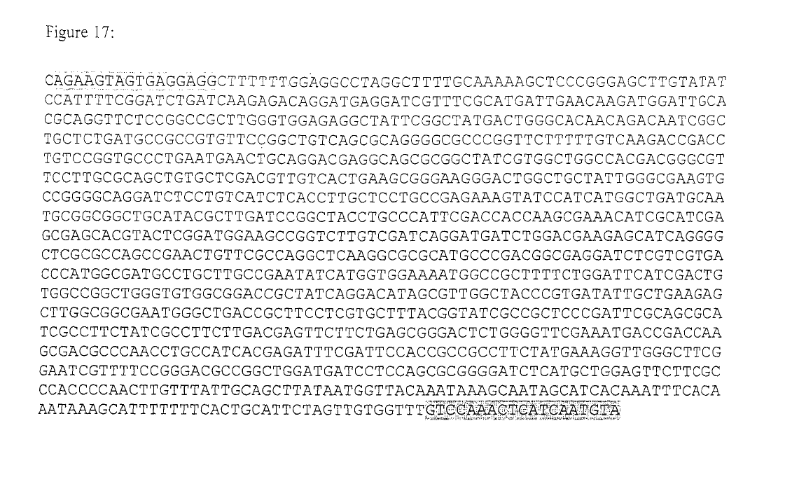

The result of RT-PCR was analyzed on the electrophoresis gel in comparison to DNA ladder to check the correctness of products sizes. The products of β-actin and GLP-1 RT-PCR were 558 bp and 250 bp respectively (FIG. 12).

Transfection of pGLP-1/Ins/pBud Plasmid

To study the insulin expression in the L cell line, the GLP-1/Ins/pBud plasmid was transfected to the extracted L cell line according, to manufacturer's protocol. Selection of stable clones was performed by replacing medium the day after transfection with complete medium, supplemented with proper amount of zeocine antibiotic that has been measured and identified in the MTT assay (500 ug/ml). Medium was changed every 2-3 days, until individual clones of the transfected cells appeared. Stable transfected cell clones were isolated for the next step analysis.

### Immunocytochemistry

The expression of the insulin protein into the L cell line was evaluated by immunocytochemistry test. In this method, mouse monoclonal antibody against human insulin as primary antibody and goat polyclonal antibody against mouse IgG conjugated with fluorescein isothiocyanate (FITC) as secondary antibody were used. The L cells were grown on 6 well tissue culture plates, containing sterilize glass coverslip before the day of transfection. After 48 h, transfected cells were fixed with 4% Paraformaldehayde. Then, the cells were incubated in 0.1% triton X-100 for permeabilization and then in 3% BSA (bovine serum albumin) for blocking. The slide was then overlaid with primary monoclonal antibody diluted at ratio 1:100 for overnight. Next, the slide was incubated with secondary antibody conjugated with FITC, diluted at 1:100 in TTBS, at RT for 2 h. Following that, the slide was incubated with DAPI nucleic acid stain to dye the nucleus of cells. Finally the slides were analyzed by an inverted phase contrast microscope with fluorescence light.

1. A method for the isolation of GLP-1 expressing cells, comprising the steps of:
a. providing a nucleic acid construct comprising the GLP-1 promoter sequence operably linked to an antibiotic resistance marker gene,
b. introducing said nucleic acid construct into a population of cells suspected to contain GLP-1 expressing cells,
c. culturing the cells of b. in the presence of an antibiotic corresponding to the antibiotic resistance marker gene,
d. selecting a cell clone that shows resistance to the antibiotic, and
e. optionally, confirming the expression of GLP-1 in the selected cell clone.

2. The method according to claim 1, wherein the GLP-1 expressing cells are L cells.

3. The method according to claim 1, wherein the population of cells suspected to contain GLP-1 expressing cells is a population of cells derived from an intestinal endocrine tumor.

4. The method according to claim 1, wherein the antibiotic is selected from the group consisting of zeocin or geneticin (neomycin).

5. The method according to claim 1, wherein the GLP-1 promoter is a rat GLP-1 promoter.

6. A nucleic acid comprising the sequence of the GLP-1 promoter, wherein said GLP-1 promoter is operable linked to an antibiotic resistance marker gene, and/or said GLP-1 promoter is operably linked to an insulin gene.

7. A population of cells isolated by a method according to claim 1, wherein the population of cells expresses GLP-1.

8. (canceled)

9. A nucleic acid according to claim 6, wherein the GLP-1 promoter is a sequence derived from a mammalian GLP-1 gene.

10. (canceled)

11. An expression vector comprising the nucleic acid according to claim 6.

12. An expression vector according to claim 11, wherein the expression vector is a mammalian expression vector.

13. A cell transformed with the nucleic acid according to claim 6.

14. The cell according to claim 13, wherein the cell is a mammalian cell.

15. The cell according to claim 13, wherein the cell is an L cell.

16. A method for the expression of insulin in a cell, comprising the steps of:
a. providing a nucleic acid construct comprising a sequence of a GLP-1 promoter operably linked to a sequence of an insulin gene, and
b. introducing said nucleic acid construct into a target cell.

17. The method of claim 16, wherein the target cell is a GLP-1 expressing cell derived from the intestine.

18. The method of claim 16, wherein the cell is a mammalian cell.

19. The method of claim 16, wherein the GLP-1 promoter comprises SEQ ID NO:1 and the insulin gene is a human insulin gene.

20. (canceled)

21. A method of producing an insulin expressing cell, wherein the method comprises the steps of a method according to claim 16.

22. (canceled)

23. A method of treatment of a subject suffering from a disease related to a disordered blood insulin level, comprising administering to the subject a therapeutically effective amount of a nucleic acid according to claim 6, or a cell comprising said nucleic acid.

24. A method of treatment according to claim 23, wherein the disease related to the disordered blood insulin level is selected from the group consisting of diabetes I, diabetes II and disorders related to nutrient metabolism.

25-26. (canceled)
